(19) United States
(12) Patent Application Publication
(10) Pub. No.: US 2008/0242557 A1
(43) Pub. Date: Oct. 2, 2008

(54) System For Detecting Molecular Interactions

(75) Inventors: Tae Kook Kim, Daejeon (KR); Yong-Weon Yi, Daejeon (KR); Jaejoon Won, Daejeon (KR)

Correspondence Address: SCHMEISER OLSEN & WATTS, 18 E UNIVERSITY DRIVE, SUITE # 101, MESA, AZ, 85201, US

(73) Assignee: CGK CO., LTD., Daejeon, KR (KR)

(21) Appl. No.: 11/572,224

(22) PCT Filed: Jul. 20, 2005
(86) PCT No.: PCT/KR05/02352

§ 371 Date: Jul. 10, 2007

(30) Foreign Application Priority Data
Jul. 20, 2004 (KR) 10-2004-0056476
Feb. 18, 2005 (KR) 10-2005-0013813

(51) Int. Cl. C40B 30/06; G01N 21/76; G01N 23/00; G01N 25/22; B01J 19/00; C40B 30/10; G01N 33/543; C12Q 1/02

(52) U.S. Cl. 506/10; 436/172; 436/57; 436/149; 436/518; 435/29; 506/12; 422/68.1

(57) ABSTRACT

This invention relates to a method for detecting interactions between molecules, such as, biomolecules, and relates to a method for screening target molecules, both in vivo and in vitro. In this invention, the first detecting material and the second detecting material are provided, wherein the first detecting material is bound to a localizer which is translocated by externally applied driving force, and the second detecting material is bound to a label. Thus, the complex of the first detecting material and the second detecting material can be detected reversibly by changing the strength of the externally applied driving force, and the target molecule can be screened effectively.

### TECHNICAL FIELD

The present invention relates to a real-time dynamic method for detecting interactions, both in vivo and in vitro, between interest molecules, such as, biomolecules. In addition, the present invention relates to a method for detecting interactions, both in vivo and in vitro, between interest molecules, such as, biomolecules and thereby screening target molecules.

### BACKGROUND ART

In general, a bioactive chemical compound means a chemical compound which binds to biomolecules, such as, protein, nucleic acid, saccharide or lipid, and controls the function or biological activity thereof, in vivo. Such a bioactive chemical compound is extracted from an organism or prepared by chemical synthesis. Various antibiotics, for example, “cyclosporine A” (Novartis AG) and “FK506” (Fujisawa), which are used for reducing immune rejection following an organ transplantation, were isolated from a microorganism, a plant or a marine organism. Such a natural or synthetic bioactive chemical compound is developed as a new drug through pharmacological activity test and through clinical test therefor, using an animal model and a human model. However, many of the natural bioactive chemical compounds are not suitable to be produced in industrial mass production system, owing to the structural complexity of the compounds and the technical restrictions in purification and isolation processes. Therefore, combinatorial chemicals and a new method of synthesis of organic chemical compound were introduced (Schreiber, S., 2000, Target-oriented and diversity-oriented organic synthesis in drug discovery, Science 287, 1964-1969).

A protein works its biological functions in vivo by binding to other proteins. The complement proteins interact with each other, and a bioactive chemical compound binds specifically to the specific portion of 3-dimensional protein structure. The interaction between two proteins strongly connotes that they are functionally related. And the bioactive chemical compound binding to a specific portion of a protein, especially relevant to diseases has potential as a drug, which diagnoses, prevents, treats or improves a disease, since the compound controls the activity of the protein. Therefore, various methods of screening and evaluating a bioactive chemical compound or a target protein as a drug candidate have been studied, wherein the methods detected the interaction between a “bait”, of which function and feature were known, and a “prey” which was an interaction partner of the bait. Until now, about 500 of target proteins relevant to diseases were identified, and 5,000 or more target proteins are expected to be identified in a couple of years.

Various studies for the roles of biomolecules, which function in metabolisms and signal transfers in a cell, have been carried out following the human genome analysis. The new research areas of chemical genomics, chemical biology and chemical proteomics, etc were introduced and they were related to the developments of various technologies of analysis design, functional genomics, proteomics, automatic engineering and bioinformatics, etc.

The methods for detecting interactions between proteins in vitro are as follows: conventional biochemical method of, for example, cross-linking, radio labeled ligand binding, X-ray crystallography and affinity chromatography, affinity blotting, immunoprecipitation and mass spectrometry (E. M. Phizicky and S. Fields, Microbiol. Rev., 59: 94-123, 1995; A. R. Mendelsohn and R. Brent, Science, 284: 1948-1950, 1999). The conventional methods mentioned above, however, require protein production and isolation processes, which require enormous efforts. In addition, since these methods should be carried out in vitro, it cannot provide the exact information about interactions in a cell.

In order to overcome the problems of the conventional methods mentioned above, functional clonings, such as, yeast two-hybrid (Y2H), yeast three-hybrid (Y3H) (Licitra, E. J., and Liu, J. O., 1996, A three-hybrid system for detecting small ligand-protein receptor interactions. Proc. Natl. Acad. Sci. USA 93, 12817-12821), drug-western (Tanaka, H., Ohima, N., and Hidaka, H. 1999. Isolation of cDNAs encoding cellular drug-binding proteins using a novel expression cloning procedure: drug-western. Mol. Pharmacol. 55, 356-363), phage display cloning (Sche, P. P., McKenzie, K. M., White, J. D., and Austin, D J. 1999, Display cloning: functional identification of natural product receptors using cDNA-phage display. Chem. Biol. 6, 707-716), fluorescence resonance energy transfer (FRET; Moshinsky D J, Ruslim L., Blake R A, Tang F., A widely applicable, high-throughput TR-FRET assay for the measurement of kinase autophosphorylation: VEGFR-2 as a prototype. J Biomol Screen. 2003 August; 8(4):447-52) and in mRNA display cloning (McPherson, M., Yang, Y., Hammond, P. W., and Kreider, B. L., 2002, Drug receptor identification from multiple tissues using cellular-derived RNA display libraries. Chem. Biol. 9, 691-698), etc. were developed.

Among the functional clonings, the yeast two-hybrid system cannot be applied to human, since the reliability of the system gets lowered and the analysis of the information obtained from the system is difficult when the genome size is larger and the genome is complex. The system should be selected in accordance with the original location of the protein in a cell. In addition, the transcription activity is affected by the library quality, and cell metabolism and cell growth. In addition, the obtained hybrid protein could be toxic.

An Y3H system is characterized in that the detection of the interaction between a bioactive chemical compound and a protein is carried out in a live cell. However, the Y3H system needs to be carried in a simple cell, such as yeast cell, like the Y2H system, which is the limitation of the system, and the system is not suitable for the detection of a a full-length membrane protein. Further, the system is not suitable to detect interactions between a bioactive chemical compound and a protein, which require posttranslational modification of the endogenous protein of yeast and require an accessory protein. Further, the yeast cell has genetically lower cell membrane permeability for a bioactive chemical compound in comparison to other mammalian cells.

A method using bioinformatics for predicting protein interaction was introduced. This method predicts interactions between proteins through molecular phylogenies or multiple sequence alignment, based on the database of genome sequence information. In addition, virtual screening methods, for example, structural genomics, molecular fingerprint and various cluster analysis method, were introduced recently.

As mentioned above, even though various methods for detecting interactions of proteins were proposed, the need for finding more effective interaction detection method has been continuous. Further, various methods for screening a bioactive chemical compound by detecting interactions of the compound and a target protein have been developed. For example, Ding, S. et. al. identified a chemical compound, which induces a Pl 9 stem cell to differentiate to a nerve cell, by screening purine molecule library using affinity chromatography (Ding, S., T. Y. H., Brinker, A., Peters, E. C., Hur, W., Gray, N. S., and Schultz, P. G. 2003, Synthetic small molecules that control stem cell fate. Proc. Natl. Acad. Sci. USA 100, 7632-7637), as a biochemical method. However, this method can be used only when the affinity is high, and in order for effective detection, numerous target proteins should be provided. Since most of the bioactive chemical compounds are hydrophobic, the compounds bind competitively to the target molecules in hydrophilic environment. Therefore, a protein would bind to a non-specific binding partner as well as a specific binding partner if an extract to be detected is applied to an agarose or other supports where a bioactive chemical compound is attached. Thus, in this method, washing process should be carried out in order to remove a binding molecule having lower affinity.

In order to overcome disadvantages of the biochemical method, genomic method was introduced. Zheng et al. used yeast three hybrid system in order to detect heterozygote and enhanced drug sensitivity in a host cell. The potent relationship between phenotype and genotype is the largest advantage of this method. That is, one hit identified using this method provides directly a gene clone to a target protein (Jaeger, S., Eriani, G., and Martin, F. 2004, Results and prospects of the yeast three-hybrid system. FEBS Lett. 566, 7-12). In this system, a novel target protein candidate can be used as a part of a fusion protein complex. This system, however, should be used only in yeast.

The chemical genomics, which uses a bioactive chemical compound in order to determine the effect of gene mutation on a cell, identifies a target active chemical compound from a group of chemical compounds or from a combinatorial library (Schreiber, S. L. 2003, The small molecule approach to biology. Chem. & Eng. 1199 News 81, 51-61; Crews, C M., and Splittgerber, U. 1999, chemical genetics: exploring and controlling cellular processes with chemical probes. Trends Biochem. Sci. 24, 317-320). In this method, hundreds or thousands of chemical compounds are arrayed with gene engineered cells in wells of a microtiter plate in order to detect interest phenotype (Clemons, P. A., Koehler, A. N., Wager. B. K., Springs, T. G., Sprig, D. R., King R. W., Schreiber. S. L., and Foley, M. A. 2001, A one-bead, one-stock solution approach to chemical genetics: part 2. Chem. Biol. 6, 71-83). For example, a report construct having a promoter inducing the expression of a GFP or a commercial marker will be included in a cell, in order to induce the expression of a certain gene or a group of genes. However, this method has also two significant problems: first, the chemical genomics is not suitable to detect a bioactive chemical compound which functions with nM unit; Secondly, only a couple of active molecules can be detected from hundreds of thousands of materials, using this method.

As mentioned above, most of the conventional technologies for detecting the interaction between proteins or for detecting the interaction between a protein and a bioactive chemical compound can be carried out only in limited kinds of cells, such as, yeast or bacteria cell. And most of the conventional technologies should be carried out in vitro. And, since the most of the conventional technologies require gene engineering and expression process, implementations of the technologies are limited by the size of DNA. And the higher false positive hit rate owing to other parameters lowers the reliability of the result.

Thus, we, inventors, studied in order to develop a novel method of real-time dynamic detection of molecular interactions in vitro and in vivo. As a result, we completed this invention by detecting and verifying the molecular interaction of bioactive molecules by monitoring the shifted label under magnetic field, following the introduction of a bioactive molecule, to which a label for the detection was attached, and following the introduction of another bioactive molecule, which was attached to a material showing magnetism or a material having magnetic material/particle to be shifted under magnetic field.

### DISCLOSURE OF INVENTION

The object of the present invention, in general, is to provide a method of real-time detection of interactions between interest molecules, such as, biomolecules, in vivo (i.e., in cell, in tissue or in body) and in vitro, and for screening target molecules. Specifically, this invention is for providing a method for directly detecting interactions, in vivo and in vitro, between “the first detecting material” of a certain biomolecule and “the second detecting material” of a biomolecule to be detected, evaluated and/or screened.

Another object of the present invention is to provide a method for directly detecting interactions, in vivo and in vitro, between “the first detecting material” and “the second detecting material”, and for screening a target molecule.

Another object of the present invention is to provide a kit or a chip for directly detecting interactions of “the first detecting material” and “the second detecting material”, in vivo and in vitro.

Another object of the present invention is to provide a method for detecting a target molecule which blocks, inhibits, activates or induces interactions between “the first detecting material” and “the second detecting material”, and for screening the target molecule.

Another object of the present invention is to provide a method for indirectly detecting interactions, in vivo and in vitro, between “the first detecting material” and “the second detecting material” by monitoring the change of cell feature or function.

### BRIEF DESCRIPTION OF DRAWINGS

FIG. 1 shows schematic figure of the present invention.

FIG. 2 is a confocal microscope picture showing translocation of nanoparticles coated with FITCs, by externally applied magnetic field, after introducing the coated nanoparticles into cells.

FIG. 3 is a confocal microscope picture showing the translocation of fluorescence in cells (red colored) expressing FKBP12-RFP proteins by externally applied magnetic field, in comparison to the cells (green colored) expressing EGFP.

FIG. 4 is a confocal microscope picture showing the translocation of the complex of FKBP12-RFP fusion protein and FK506-MNP in a cell by externally applied reversible magnetic field, wherein the FK506-MNP is the pharmaceutical binding partner of the FKBP12-RFP fusion protein.

FIG. 5 is a confocal microscope picture showing the translocation of the cells (indicated with red arrow) expressing mRFP-Cascase-3s while changing the strength of external magnetic field.

FIG. 6 is a confocal microscope picture showing the translocation of the complex of IκBα protein and ReIA protein by externally applied reversible magnetic field.

FIG. 7 is a confocal microscope picture showing the translocation of the complex of anti-phospho-IκBα antibody and IκBα protein by externally applied reversible magnetic field, as the result of detection of phosphorylated IκBα protein using anti-phospho-IκBα antibody.

FIG. 8 is a confocal microscope picture showing the translocation of the complex of UAS oligonucleotide and GAL4 protein by externally applied reversible magnetic field.

FIG. 9 is a conceptual picture showing the formation of the complex resulted from the interaction between miRNA of let-7b and mRNA of lin-28, and showing the translocation thereof.

FIG. 10 is a confocal microscope picture showing the translocation of the complex of miRNA and mRNA by externally applied reversible magnetic field.

FIG. 11 is a confocal microscope picture showing the translocation of the complex of β-CD and MBP by externally applied reversible magnetic field.

FIG. 12 is a conceptual picture showing assay of binding partner protein with microarrayed cells.

FIG. 13 is a schematic view of IκBα-mRFP, IκBα-ECFP-FKBP 12, EYFP-ReIA and mRFP-β TrCP fusion proteins.

FIG. 14 is a schematic view showing the interaction of IκBα-mRFP and nano-particles by the use of anti-phospho-IκBα (Ser32) antibody.

FIG. 15 is a confocal microscope picture showing the translocation of the nano-particle complex, which was resulted from TNF-α treatment as in FIG. 14, by externally applied reversible magnetic field.

FIG. 16 is a schematic view showing the formation of nanoparticle complex of proteins of FIG. 13, after the proteins were treated with TNF-α.

FIG. 17 is a confocal microscope picture showing the translocation of the nano-particle complex of FIG. 16 by externally applied reversible magnetic field.

FIG. 18 shows a retrovirus construct for preparing expression library.

FIG. 19 shows general structures of FK506 and FK506-biotin, which were used for the identification of target molecules.

FIG. 20 is a confocal microscope picture showing specifically localized EGFP fusion protein by externally applied magnetic field. It was verified that the specific fusion proteins from the expression library were FKBP 12 and FKBP52 which were the targets of the FK506.

FIG. 21 shows general structure of CGK1026.

FIG. 22 shows that the CGK1026 inhibits the activity of HDAC protein.

FIG. 23 is a confocal microscope picture showing competitive inhibition of the combination between Biotin-FK506 and FKBP 12 by the FK506 treatment.

FIG. 24 is a confocal microscope picture showing that WT inhibits the interaction between ATM phosphorylation enzyme and CGK606.

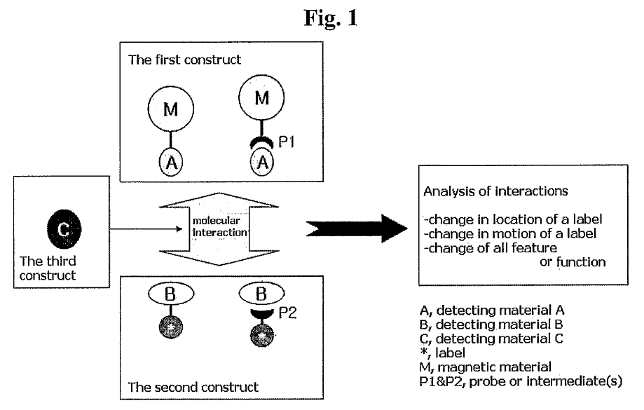

### MODE FOR THE INVENTION

In order to achieve the above-mentioned purposes of the present invention, we, inventors, designed two constructs. More specifically, the first construct comprises a localizer, which is translocated by an externally applied driving field, and the first detecting material, for example, a biomolecule to be analyzed, wherein the first detecting material is attached to the localizer directly or indirectly by the use of, for example, a linker. The first detecting material of biomolecule and the localizer can be provided as a fused form. Further, the first construct can be provided as two or more fragments, which are formed by the indirect binding of at least one of the first detecting materials of biomolecules and localizers to which probes are fused to help to recognize the biomolecules. The second construct includes one or more second detecting materials, such as biomolecules, to which at least one label is attached for detection. In the second construct, the label and the biomolecule to be analyzed can be provided in a directly fused form. Further, the second construct can be provided as two or more fragments, which are formed by an indirect binding of at least one of the biomolecules and labels to which the probes are fused to help recognize the biomolecules. A real-time dynamic detection of the complex formed by the interaction of the first detecting material and the second detecting material is carried out by approaching them near enough to interact with each other in the same field or system, followed by detecting the label which has changed in location or motion with a suitable apparatus. Further, in this invention, it is possible to indirectly detect interactions by monitoring the change of cellular morphology or function after applying an external driving force. In this invention, if the first detecting material is working as a “bait”, then the second detecting material is working as a “prey”, and vice verse. In the present invention, the change of the location or motion can be monitored by the use of, for example, optical method employing, such as, microscope, and can be monitored by a scanner, radioactive label detecting device, fluorescence polarization reader (FP reader), spectrophotometer, MRI (magnetic resonance imaging), SQUID, fluorescence detector and luminescence detector, etc.

Specifically, the method for detecting molecular interactions of the present invention comprises the steps of: i) providing the first construct of a binding complex of the first detecting material and a localizer which is translocated by an externally applied driving force; ii) providing the second construct comprising the second detecting material to which a label is attached for detection; iii) approaching the first construct and the second construct, in the same field or system, near enough to interact with each other; and iv) applying an external driving force followed by detecting the label which has changed in location or motion.

Further, the method for screening a target molecule of the present invention comprises the steps of: i) providing the first construct of binding complex of the first detecting material and a localizer which is translocated by externally applied driving force; ii) providing the second construct comprising the second detecting material to which a label is attached for detection; iii) approaching the first construct and the second construct, in the same field or system, near enough to interact with each other; and iv) applying external driving force followed by detecting the label changed in location or motion; and v) isolating and identifying the molecule from the first construct or the second construct. The screening of the target molecule can be further carried out using a conventional method, for example, RT-PCR, genomic DNA PCR or using Mass Spectroscopy.

Further, in accordance with the present invention, three constructs are provided in order to detect interactions among the molecules and to screen a target molecule. Specifically, the method of the present invention comprises the steps of: i) providing the first construct of a binding complex consisting of the first detecting material and a localizer which is translocated by an externally applied driving force; ii) providing the second construct comprising the second detecting material to which a label is attached for detection; iii) providing the third construct comprising a material to be detected; iv) approaching the three constructs, in the same field or system, near enough to interact with each other; v) applying external driving force followed by detecting the label having changed in location or motion; and vi) isolating and identifying the molecule from the first construct, the second construct, or the third construct. The screening of the target molecules can be further carried out using a conventional method, for example, RT-PCR, genomic DNA PCR or using a Mass Spectroscopy.

In the present invention, the molecule to be detected in the first construct, the second construct or the third construct can be provided as a library form.

In one embodiment, the present invention employs a magnetic field as an externally applied driving force. As a localizer of the first construct, a nanoparticle (preferably 5 nm˜2,000 nm in diameter), is used, wherein a molecule, for example, a biomolecule, is bound to or coated on the localizer. The localizer can be bound to or coated by two or more molecules. The second construct comprises an other biomolecule of a binding partner, which interacts with the biomolecule of the first construct, wherein a label is attached to the molecule of the second construct for detection. The interest target molecule can be detected real-time after the first construct and the second construct are introduced into the same cell followed by applying a magnetic field as a driving force and by determining the translocated label. Further, in the present invention, reversible detection can be carried out by changing the strength of the magnetic field. And the comparison detection is carried out using a negative control that does not show any change in location or motion, or using a positive control that shows change in location or motion, while varying the strength of the external magnetic field.

In another embodiment, the present invention comprises the steps of: i) providing a magnetic particle coated with certain bioactive chemical compounds, ii) introducing the coated magnetic particle of i) into a cell containing a target molecule to which a label is attached; iii) applying a magnetic field to the cell of ii) in order to translocate the magnetic particle in the cell; iv) monitoring the location or motion of the label; and v) isolating and identifying the target molecule from the cell in which the label has changed in location or motion.

When carrying out this invention in vivo, it can be carried out: for example, in prokaryote or eukaryote; in an organism, tissues or cells of mammals; and in an organism, tissues or cells of plants. Particularly, the method of this invention can be carried out in an organism, tissues or cells of Zebra fish, C-elegans, Yeast, Fly or Frog. The first construct, the second construct and the third construct are introduced into a cell by the use of, for example, transducible peptide (or fusogenic peptide), lipid (or liposome) or the binding complex thereof; or by incubating the constructs with a cell in Opti-MEM; or by electroporation or magnetofection. In particular, when carrying out this invention in a live cell, this invention can be carried out within living cells on a culture plate/dish or within microarrayed live cells. Further, microarrayed cells can be employed for this invention.

The externally applied driving force in the present invention depends on the feature, such as, the physical, chemical and biological feature, of the localizer of the first construct. For example, the external driving force can be applied with an optical device, such as, optical tweezer.

In the present invention, the “biomolecule” is understood as including all the materials, which represent biological activities in vivo, such as, nucleic acid, mono-/oligo-/poly-nucleotide, protein, mono-/oligo-/polypeptide, amino acid, mono-/oligo-/poly-saccharide, lipid, vitamin, chemical compound and the materials constructing thereof.

In the present invention, the “bait” means a biomolecule used for detecting interaction with other biomolecule.

In the present invention, the “prey” means a biomolecule to be detected or screened, which is an interaction partner of the “bait”.

In the present invention, a “target molecule” means the prey, which interacts with the bait. Also, the target molecule means all the materials to be identified, which activate or induce the interactions between the bait and the prey or which block or inhibit the interactions between the bait and the prey.

In the present invention, a “localizer” means the material which is translocated, shifted or moved in the direction according to the externally applying driving force.

In the present invention, the externally applied driving force means all types of forces which result in a translocation or motion of the localizer defined above. The external driving force includes, for example, electric force, electro-magnetic force, magnetic force and gravity, etc.

In the present invention, the label of the second construct comprises a fluorescent material, which emits fluorescence by itself or develops fluorescence by the interaction with the first construct or other materials, for example, fluorescent dye, such as, FITC and rhodamine, etc; fluorescent protein, such as, GFP, YFP, CFP and RFP, etc.; tetracystein motif; and fluorescen nanoparticle. In the present invention, the label of the second construct comprises a luminescent material, which emits luminescence by itself or develops luminescence by the interaction with the first construct or other materials, for example, luciferase. As a radio-active label, 32P, 35S5 3H and 14C, etc. can be used in the present invention.

In the present invention, a binding between a bioactive molecule and a localizer includes, for example, a physical, chemical electrostatic and biological direct or indirect binding. The bioactive molecule can be coated on the surface of the localizer or label.

In the present invention, the probe of the first construct or the second construct, which is used for detection, comprises, for example, an antibody, protein, protein domain, protein motif and peptide, etc. The probe, which binds to the biomolecule by a chemical, physical, biological or electrostatic binding, can combine the detecting material and the localizer or combine the biomolecule and the label.

Preferred embodiments of this invention are described in the following examples. Other embodiments within the scope of the claims herein will be apparent to those skilled in the art from consideration of the specification or practice of the invention as disclosed herein. It is intended that the specification, together with the examples, be considered exemplary only, with the scope and the spirit of the invention being indicated by the claims which follow the examples. The examples herein are meant to exemplify the various aspects of carrying out the invention and not intended to limit the scope of the invention in any way. The examples do not include detailed descriptions of conventional methods employed. Such methods are well-know to those skilled in the art and are described in numerous publications. In addition, all the publications referred herein are integrated hereto as references.

### EXAMPLES

### Example 1

### Determination of the Localization in Cells Using Fluorescent FITC Coated Nano-Particles

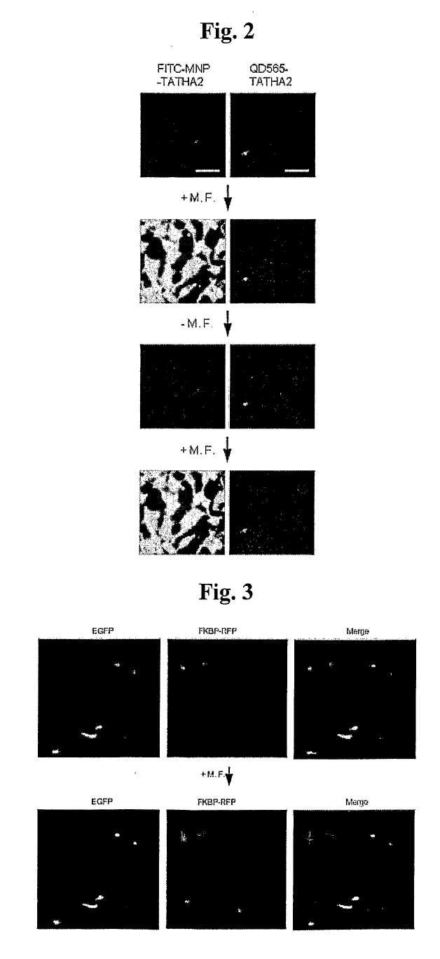

FITC-MNP-TATHA2 complex, FITC coated superparamagnetic nanoparticles (MNPs), was prepared by reacting a streptavidin conjugated superparamagnetic nanoparticle (50 nm; streptavidin nanoparticles) with a FITC-biotin and TATHA-biotin. The complex TATHA2 peptide was synthesized by the conventional method (Wadia, et al., Nat Med. 10:310-315 (2004)), and the N-terminal thereof was labeled with a biotin. As a negative control, QD565-TATHA2 was prepared by reacting the nanoparticle of QD565 and TATHA2-biotin, wherein the QD565 would not change its location by the externally applied magnetic field. HeLa cell (available from ATTC) was rinsed with a serum free DMEM. Then, the rinsed cell was incubated in Opti-MEM (Invitrogen) at 37° C. for 12 hrs in a CO2 incubator, after the cell was treated using a mixed solution of FITC-MNP-TATHA2 complex and QD565-TATHA2 complex. After transferring the cell onto a focal plate of a confocal microscope, a magnetic field was applied thereto from the bottom of the plate with a commercially available paramagnet or electromagnet. The fluorescence protein translocated by the applied magnetic field was detected after focusing the focal plate of the confocal microscope (FIG. 2). It was observed only in the cell containing FITC-MNP-TATHA2 that the FITC-MNP-TATHA2 complex was translocated by the externally applied driving force, while the translocation was not observed in the cell containing a QD565-TATHA2 of negative control.

### Example 2

### Detection of the Interactions of Various Biomolecules in a Cell

1) Detection of the Interaction in a Cell Using a Fusion Protein of Fluorescent Protein and a Nano-Particle Coated with Bioactive Chemical Compound:

Superparamagnetic nanoparticles (MNPS) were coated with FK506 by reacting Streptavidin MicroBeads (50 run in diameter, Streptavidin MicroBeads, Miltenyi Biotec) and FK506-biotin. A HeLa cell, which expresses EGFP protein or a fusion protein of RFP-FKBP, was rinsed with a serum free DMEM. Then, the rinsed cell was incubated in a mixed medium comprising a FK506 coated MNP and Opti-MEM (Invitrogen) at 37° C., for 12 hrs in a CO₂ incubator in order to introduce a FK506 coated MNP into the HeLa cell. After transferring the cell onto the focal plate of confocal microscope, a magnetic field was applied thereto from the bottom of the plate with a paramagnet or electromagnet. The fluorescence protein translocated by the applied magnetic field was detected while controlling the focal plate of the confocal microscope (FIG. 3). Only the cells (red-colored) expressing a RFP fused FKBP 12 were localized by the externally applied driving force, while the EGFP expressing cells (green-colored), negative controls, were not translocated.

2) Detection of the Interaction in a Cell Between FK506 and its Pharmaceutical Binding Partner of FKBP12FK506 Binding Protein 12)

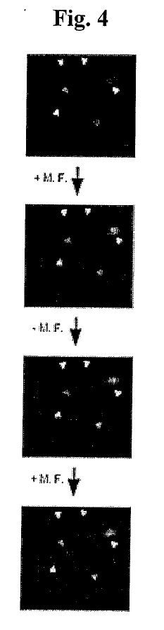

A FK506-MNPS complex was obtained by reacting Streptavidin MicroBeads (Miltenyi Biotec), FK506-biotin and TATHA2-biotin at 40 C for 1 hr while agitating the reaction mixture. Unbound ligands were removed from the reaction mixture by purification using a Captivate magnetic separator (Molecular probes). After introducing RFP-fused FKBP 12 or EGFP expressing plasmid into a HeLa cell (ATCC), the cell was incubated. Then, the FK506-coated MNPs was introduced into the cell, followed by a rinsing of the HeLa cell with a serum free DMEM. Next, the magnetic field (M.F.) was externally applied to the cell using Magnetofector (commercially available from Chemicell) or permanent magnetic material. The confocal images for the live cells were taken using a confocal laser microscope while applying a magnetic field as well as not applying a magnetic field. In FIG. 4, only the cell (indicated with red arrow) expressing RFP-fused FKBP 12 was localized by the external magnetic field, while the EGFP expressing cell (indicated with green arrow), a negative control, did not show any motion.

3) Detection of the Interaction in a Cell Between Caspase-3 Inhibitor Peptide and its Binding Partner of Caspase-3:

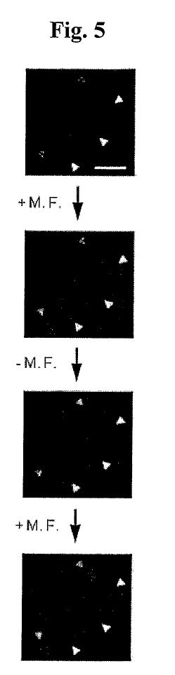

A DEVD-MNPs complex was obtained by reacting a Streptavidin nanoparticle, caspase-3 inhibitor II (DEVD)-biotin conjugate (CALBIOCHEM) and TATHA2-biotin at 40 C for 1 hr while agitating the reaction mixture. Unbound ligands were removed from the reaction mixture by purifying the complex using a paramagnet. After introducing mRFP-Caspase3 or EGFP expressing plasmid into a HeLa cell (ATCC), the cell was incubated. The DEVD-MNPs were introduced into the cell, and a magnetic field (M.F.) was externally applied to the cell using a paramagnet or electromagnet. The confocal images were captured using a confocal laser microscope (Carl Zeiss) for the live cells while applying magnetic field as well as not applying magnetic field. In FIG. 5, only the cells (indicated with red arrows) expressing mRFP-Caspase3 were localized by the external magnetic force, while the EGFP expressing cells (indicated green arrows), negative controls, did not show any motion.

4) Detection of the Interaction in a Cell Between IκBα Protein and its Binding Partner of ReIA Protein:

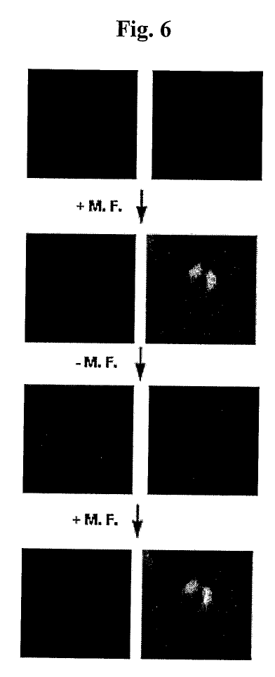

A FK506-MNPs complex was obtained by reacting a Streptavidin nano-particle, FK506-biotin and TATHA2-biotin at 40 C for 1 hr while agitating the reaction mixture. Unbound ligands were removed from the reaction mixture by purifying the complex using a paramagnet. After introducing ECFP and FKBP 12 fused IκBα, protein (IκBα-ECFP-FKBP 12) and EYFP fused ReIA (EYFP-ReIA) expressing plasmids into a HeLa cell (ATCC), FK506-MNPs were introduced into the cell, and a magnetic field (M.F.) was externally applied to the cell using a paramagnet or electromagnet. The confocal images were taken using a confocal laser microscope (Carl Zeiss) for the live cells while applying a magnetic field as well as not applying magnetic field. In FIG. 6, IκBα-ECFP-FKBP 12 fusion protein and EYFP-ReIA fusion protein were translocated by the external magnetic force.

5) Detection of the Interaction in a Cell Between IκBα Protein and its Antibody of Anti-Phospho-IκBα

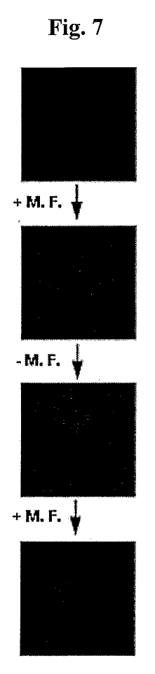

An anti-phospho-IκBα-MNPs complex was obtained by reacting a Streptavidin nano-particle, anti-phospho-IκBα-biotin and TATHA2-biotin at 40 C for 1 hr, while agitating the reaction mixture. Unbound ligands were removed from the reaction mixture by purifying the complex using a paramagnet. After introducing mRFP fused IκBα (IκBα-mRFP) expressing plasmids into a HeLa cell (ATCC), the anti-phospho-IκBα-MNPs were introduced into the cell and a magnetic field (M.F.) was externally applied to the cells using a paramagnet or electromagnet following TNF-α treatment for 5 minutes. Confocal images were taken using a confocal laser microscope (Carl Zeiss) for the live cells while applying a magnetic field as well as not applying magnetic field (FIG. 7).

6) Detection of the Interaction in a Cell Between GAL4 Protein and Oligonucleotide (“UAS) Having GAL Binding Part:

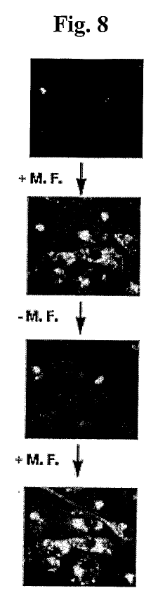

The oligonucleotide having UAS DNA sequence (SEQ ID No.: 1) to which a GAL4 protein of yeast transcription factor can bind, biotin-CCCAGTTTCTAGACGGAG (UAS-biotin) was synthesized. A UAS-MNPs complex was obtained by reacting a Streptavidin nanoparticle, UAS-biotin and TATHA2-biotin at 40 C for 1 hr, while agitating the reaction mixture. Unbound ligands were removed from the reaction mixture by purifying the complex using a paramagnet. After introducing EGFP fused GAL4 (GAL4-EGFP) expressing plasmids into a Hela cell (ATCC), a magnetic field (M.F.) was externally applied to the cell using a paramagnet or electromagnet following the introduction of UAS-MNPs into the cell. The confocal images were taken using confocal laser microscope (Carl Zeiss) for the live cells while applying a magnetic field as well as not applying magnetic field (FIG. 8).

7) Detection of the Interaction in a Cell Between miRNA and mRNA:

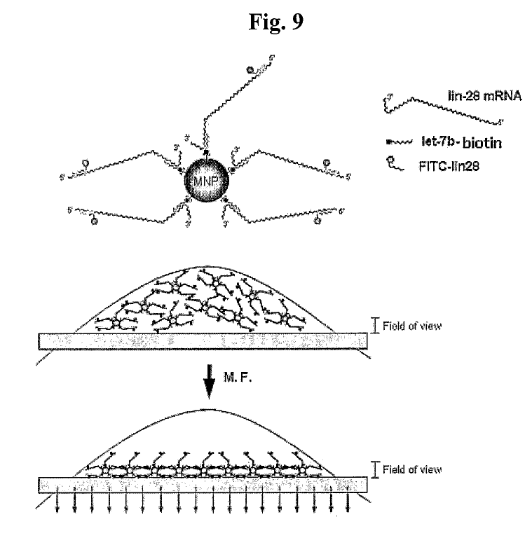

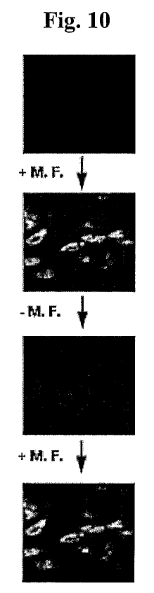

In order to detect an interaction between miRNA and mRNA which is a target of the miRNA, a let-7b miRNA (SEQ ID. No.: 2), which binds to lin-28 mRNA, was selected (FIG. 9). A let-7b miRNA was synthesized as UGAGGUAGUAGGUUGUGUGGUU-biotin (let-7b-biotin), and FITC-CCCTATAGTGAGTCGTATTA(FITC lin28p) was synthesized in order to label lin-28 mRNA (SEQ ID. No.: 3). The let7b-MNPs complex was obtained by reacting Streptavidin nano-particles, let-7b-biotin and TATHA2-biotin at 4° C. for 1 hr, while agitating the reaction mixture. Unbound ligands were removed from the reaction mixture by purifying the complex using a paramagnet. After introducing lin-28 mRNA expressing plasmids into a HeLa cell (ATCC), magnetic field (M.F.) was externally applied to the cells using a paramagnet or electromagnet following the introduction of let7b-MNPs and FITC-lin28p into the cell. The confocal images were taken using a confocal laser microscope (Carl Zeiss) for live cells while applying a magnetic field as well as not applying magnetic field (FIG. 10).

8) Detection of the Interaction in a Cell Between β-Cyclodextrin and MBP Protein:

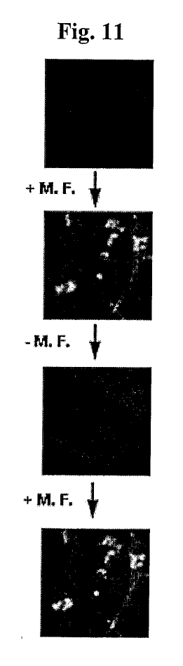

β-cyclodextrin (Sigma) was biotinated using an EZ-Link NHS-PEO Solid Phase Biotinylation kit (Pierce) (βCD-biotin). βCD-MNPs complex was obtained by reacting a Streptavidin, nanoparticle, βCD-biotin and TATHA2-biotin at 40 C for 1 hr, while agitating the reaction mixture. Unbound ligands were removed from the reaction mixture by purifying the complex using a paramagnet. After introducing EGFP fused MBP (MBP-EGFP) expressing plasmids into a HeLa cell (ATCC), the βCD-MNPs were introduced into the cell and a magnetic field (M.F.) was externally applied to the cell using paramagnet or electromagnet. The confocal images were taken using a confocal laser microscope (Carl Zeiss) for the live cells while applying a magnetic field as well as not applying magnetic field (FIG. 11).

### Example 3

### Detection of Binding Partner Proteins in Micro-Arrayed Cells

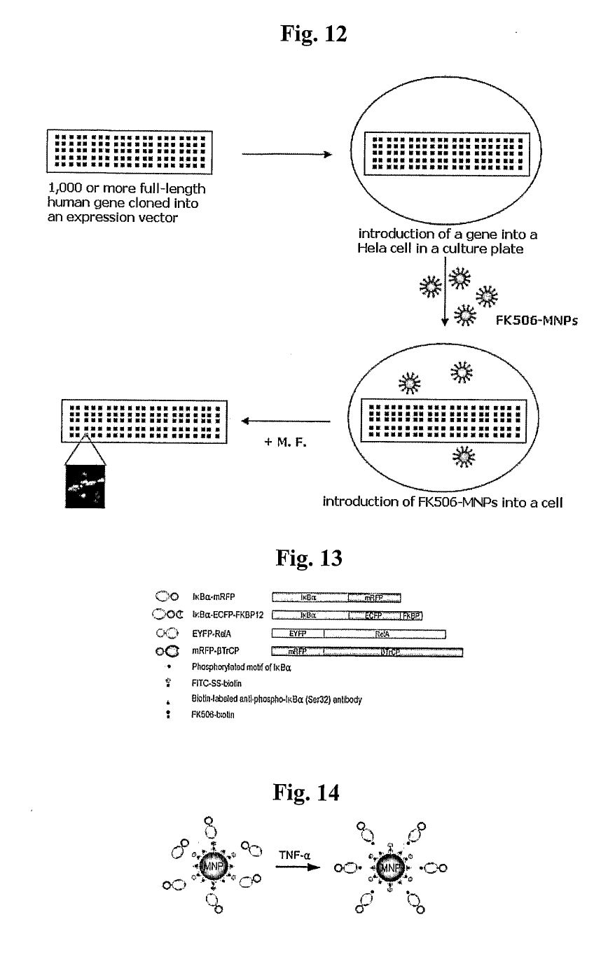

A number of about 1,000 full length ORFs (open reading frames) genes originated from human were distributed from The Center for Functional Analysis of Human Genome (Korea). 1,000 or more genes were proliferated in E. coli, and plasmid DNAs were extracted. Then, the extracted DNAs were cloned into a pcDNA-GFP-DEST vector (Invitrogen) in order to express the genes in an animal cell. The genes inserted into the pcDNA-GFP-DEST vector would express a fusion protein formed by the fusion of a GFP protein to C-terminal of the interest protein. The microarray of cells was prepared by a conventional method [see Ziauddin and Sabatini, Nature 411 : 107-110 (2001)], and was verified by the expression of the fused GFP. The FK506-MNPs prepared according to the example 2 were introduced into the microarrayed cells, and a magnetic field (M.F.) was externally applied to the cells using a paramagnet or electromagnet. The confocal images were taken using a confocal laser microscope (Carl Zeiss) for the live cells while applying a magnetic field as well as not applying magnetic field. An RT-PCR and mRNA analysis for the positive clone verified that the positive clone Was FKBP 12 which was a pharmaceutically relevant target for FK506 (FIG. 12).

### Example 4

### Detection of Post Translation Modification and Change of Protein Complex by a Signal Transduction

With a TNF-α/IκBα signal transduction system, a post translation modification and a change of protein complex were detected. A IκBα protein regulates various signals by forming complex with a NF-K B protein such as ReIA/p65, p50 and c-ReI, etc. If a cell is treated with TNF-α, the 32th serine and the 36th serine of an IκBα are phosphorylated by the increase of IκBα kinase (IKK) activity, and thereby the IκBα protein and the β TrCP protein are combined [Brown. et al., Science 267: 1485, (1995)]. Thus, in order to detect the phosphorylation of IκBα by a TNF-α treatment and the formation of IκBα-βTrCP complex, an expression construct was prepared as follows (FIG. 13). An expression vector was designed by fusing an IκBα gene and an mRFP gene in order to express an IκBα-mRFP fusion protein. In addition, an expression vector was designed by fusing an IκBα gene, ECFP gene and FKBP 12 gene in order to express IκBα-ECFP-FKBP 12 fusion protein. Further, an expression vector was designed by fusing an EyFP gene and an ReIA gene in order to express an EYFP-ReIA fusion protein in a cell. And an mRFP-β TrCP fusion protein was obtained with the use of expression vector containing the respective genes in fusion. An anti-phospho-IκBα antibody (Cell Signaling Technology) was biotinated using an EZ-Link NHS-PEO Solid Phase Biotinylation kit (Pierce), and western blot was carried out.

1) Detection of Phosphorylation of Protein by a Signal Transduction:

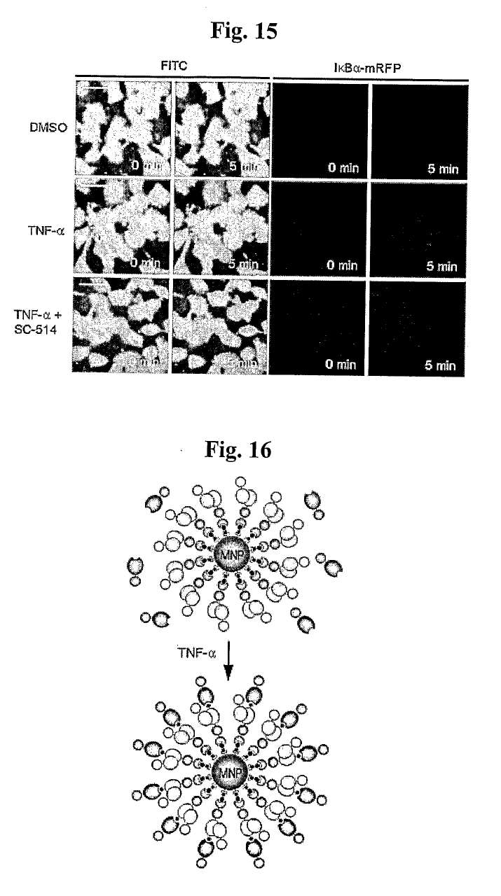

An IκBα (Ser32)-MNPs complex was obtained by reacting Streptavidin nanoparticle, biotin-SS-FITC, biotin-TATHA2 and biotin-anti-phospho-IκBα (Ser32) antibody at 4° C. for 1 hr, while agitating the reaction mixture. After further introducing an IκBα (Ser32)-MNPs complex into a HeLa cell (ATCC) according to example 1 where an IκBα-mRFP expression vector is introduced, the cells were treated with 10 ng/ml of TNF-α for 5 minutes. Confocal images were taken using a confocal laser microscope (Carl Zeiss) for the live cells while applying a magnetic field as well as not applying magnetic field (FIGS. 14 and 15). 1 mM of SC514 was used as an IKK inhibitor. The phosphorylated IκBα by the TNF-α treatment was combined with an IκBα(Ser32)-MNPs complex, and was translocated by an externally applied magnetic force. On the other hand, when a SC-514 of IKK inhibitor was treated, no translocation was observed since the IκBα phosphorylation had not occurred.

2) In Order to Detect a Protein Complex Formation by a Signal Transduction, the Following Experiment was Carried Out:

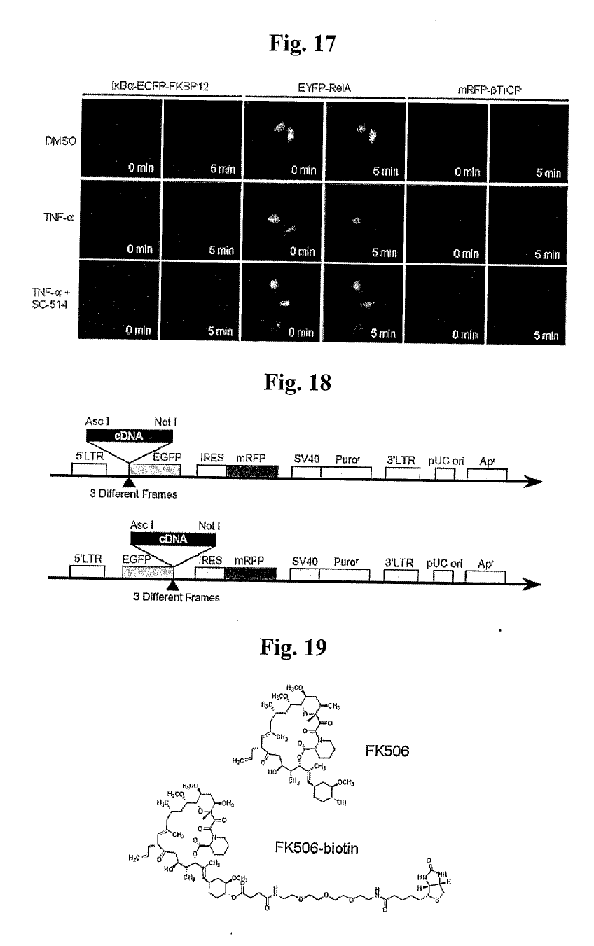

After introducing an IκBα-ECFP-FKBP 12 fusion protein expression vector, EYFP-ReIA fusion protein expression vector, and mRFP-β TrCP fusion protein expression vector into a HeLa cell at the same time, a FK506-MNPs complex obtained in the example 2 was introduced into the cell. The cell was treated with 10 ng/ml of TNF-α for 5 minutes. Confocal images were taken using a confocal laser microscope for the live cells while applying a magnetic field as well as not applying magnetic field (FIGS. 16 and 17). Regardless of the signal transduction, an IκBα-ECFP-FKBP 12 fusion protein and an EYFP-ReIA fusion protein were translocated by an externally applied magnetic force. However, the mRFP-β TrCP fusion protein, which binds to a phosphorylated IκBα by signal transduction, was translocated only when treated with TNF-α. A SC-514 of IKK inhibitor inhibited combining of an IκBα-ECFP-FKBP 12 fusion protein and a mRFP-β TrCP fusion protein, while it did not inhibit the interaction between an IκBα-ECFP-FKBP 12 fusion protein and an EYFP-ReIA fusion protein.

### Example 5

### Screening, Detection and Assay of the Interaction in Cells Between Bioactive Molecule and its Binding Partner Protein from Genome Level cDNA Expression Library

1) Screening, Detection and Assay of the Interaction in a Cell Between FK506 and its Binding Partner Protein from a Genome Level cDNA Expression Library:

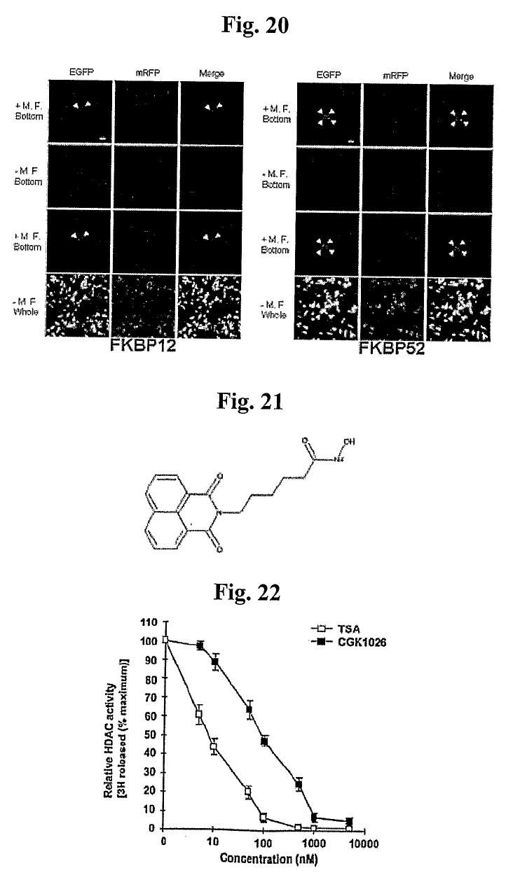

A retrovirus construct for preparing an expression library was made. The pBabe-puro vector depicted in FIG. 18 was used as the backbone [Morgenstern, et al., Nucleic Acids Res. 18:3587-3596 (1990)]. The bicistronic mRNA, which was transcripted from 5′LTR and has an EGFP fusion protein and the mRFP, was expressed from the constructed retrovirus. In order for the insert to be introduced into in-frame of EGFP at the largest amount, three types of frames were designed (FIG. 18). A random cDNA fusion to 3′ or 5′terminal of an EGFP was carried out in accordance with a conventional method (Escobar, et al., Plant Cell 15:1507-1523 (2003)). After synthesizing a FK506-biotin having a structure as depicted in FIG. 19 in accordance with the conventional method (McPherson, et al., Chem Biol. 9:691-698 (2002)), FK506-biotin was verified using MALDI MS. A FK506-MNPs complex was obtained by reacting a Streptavidin nanoparticle, FK506-biotin and TATHA2-biotin at 40 C for 1 hr. An EGFP-fusion protein expression library was prepared using Jurkat cells following a conventional method [Escobar, et al., Plant Cell 15:1507-1523 (2003)]. Infectious retrovirus was prepared by transfecting the packaging cell (Phoenix-ampho) (from G. P Nolan, Stanford University, USA) with a retrovirus expression library. Supernatant was removed from the transfected packing cells after 48 hrs from the transfection, and the removed supernatant was used for infecting HeLa cells, after the supernatant was filtered with 0.45 μm (Millipore). The HeLa cells were infected with virus supernatant to which 4 μg/ml of polybrene was added, 2 days after the infection, 1 μg/ml of puromycin was introduced into the infected cell, and the cell was cultured selectively for 3 days. Next, FK506-MNPs prepared as mentioned above were transferred into the cell as disclosed in example 1, and a magnetic field (M.F.) was externally applied to the cell using a paramagnet or electromagnet. The confocal images were taken using a confocal laser microscope for the live cells while applying a magnetic field as well as not applying a magnetic field. The mRFP, which was expressed at the same time with EGFP, was monitored as a false positive signal. In FIG. 20, an arrow means a positive clone. The sequence analysis of RT-PCT product from mRNA the positive clones showed that the positive clones were FKBP 12 and FKBP52 which were pharmaceutically relevant targets for FK506. A sequence analysis and RT-PCR for the positive clones were carried out following the conventional methods [Escobar, et al., Plant Cell 15:1507-1523 (2003)].

2) Screening, Detection and Assay of the Interaction in Cells Between CGK1026 and its Binding Partner Protein from a Genome Level cDNA Expression Library:

CGK1026 was verified as a biomolecule that enhances the activity of human telomerase [FIG. 21; Won, et al., PNAS, 101: 11328-11333 (2004)]. A CGK1026-biotin derivative was prepared in order to screen a protein which binds to CGK1026. A biotin derivative was obtained by reacting a Streptavidin nanoparticle with CGK1026-biotin and TATHA2-biotin at 4° C. for 1 hr, while agitating the reaction mixture. The protein, which binds to CGK1026, was verified as HDAC using retrovirus library. The CGK1026 inhibited HDAC activity in a similar level in comparison to the well-known HDAC inhibitor of TSA (Won, et al., PNAS, 101: 11328-11333 (2004)) (FIG. 22).

### Example 6

### Drug Screening of Interaction Inhibitor and Activator

1) Detection of Interaction Activator:

An IκBα-mRFP fusion protein expression vector and an IκBα (Ser32)-MNPs complex were introduced into HeLa cells as disclosed in 1) of example 4, and the cells were treated with various chemical compounds and growth factors. Then, a localization of an IκBα-mRFP fusion protein was detected with a confocal microscope while applying a magnetic field. As a result, only TNF-α, IL-2, LPS and Fas were observed to translocate the IκBα-mRFP fusion protein under the magnetic field (FIGS. 15 and 17).

2) Inhibition of Interaction by FK506:

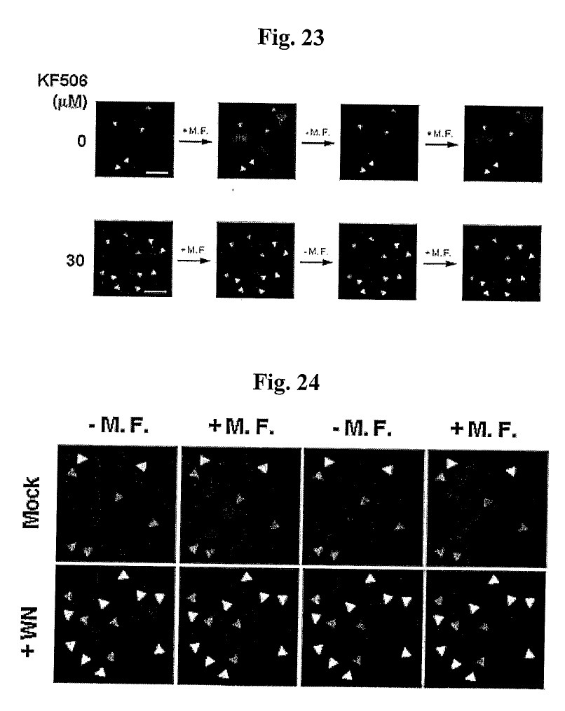

After introducing a FK506-MNPs complex and a FKBP12-mRFP expression vector into a cell as disclosed in example 3, the cell was treated with various chemical compounds, and afterward, the translocation was detected. Among the treated compounds, only FK506 was observed as competitively inhibiting the translocation of a FK-BP 12-mRFP fusion protein (FIG. 23).

3) Detection of Interaction Inhibitor:

GK606 was verified as an inhibitor to ATM (ataxia telangiectasia) phosphorylase. A CGK606-MNPs complex was obtained by coating a nanoparticle with CGK606 and TATHA2. After introducing an ATM-mRFP fusion protein expressing vector into a HeLa cell, CGK606-MNPs were introduced into the cell as disclosed above. After treating the cell with various bioactive low molecular weight chemical compounds, the translocation of ATM-mRFP fusion protein by an applied driving force was observed with a confocal microscope. Among the bioactive molecules, only a wortmannin inhibited the localization of an ATM-mRFP fusion protein by an applied magnetic field (FIG. 24).

### INDUSTRIAL APPLICABILITY

As mentioned above, a real-time dynamic detection of the complex formed by the interaction is carried out by approaching i) the first construct of a biomolecule and a localizer which is translocated by externally applied driving force; and ii) the second construct comprising other biomolecule to which a label is attached for detection in the same field or system according to the invention. Translocation of the complex also can be determined dynamically while varying the strength of externally applied driving force, thus, it is reversible. Therefore, the present invention can be used in discovering a novel pharmaceutical use from existing drugs and improving the pharmaceutical activities of the existing drugs as well as in detecting and screening a target protein or a new drug candidate, without destructing and screening a target protein or a new drug candidate, without destructing a cell and without the limitation of the size of the target molecule to be detected, in vivo and in vitro.

1. A method for detecting molecular interactions comprising:
i) providing a first construct comprising a first detecting material and a localizer which is translocated by an externally applied driving force;
ii) providing a second construct comprising a second detecting material to which a label is attached for detection;
iii) locating the first construct and the second construct, in the same field or system, near enough to interact with each other; and
iv) applying an external driving force, and detecting if the label has changed in its location or motion according to the interaction.

2. The method of claim 1, wherein the material is a bioactive molecule.

3. The method of claim 2, wherein the bioactive molecule is one or more molecules selected from the group consisting of nucleic acids, nucleotides, proteins, peptides, amino acids, saccharides, lipids, chemical compounds, and the constructing material thereof.

4. The method of claim 1, wherein the externally applied driving force is a magnetic field.

5. The method of claim 4, wherein the magnetic field is an electro magnetic or paramagnetic field.

6. The method of claim 4, wherein the detection is carried out by changing the strength of the magnetic field.

7. The method of claim 4, wherein the localizer includes a magnetic material.

8. The method of claim 7, wherein the size of the localizer is between 5 nm and 2,000 nm in diameter.

9. The method of claim 1, wherein the label is a radioactive, fluorescent or luminescent material.

10. The method of claim 9, wherein the fluorescent material or the luminescent material emits fluorescence or luminescence by itself, or develops fluorescence or luminescence when it binds to the molecule of the first construct or another molecule.

11. The method of claim 10, wherein the fluorescent material is a fluorescent dye, or a tetracystein fluorescent motif, or a fluorescent protein such as GFP, YFP, CFP or RFP, or a fluorescent nano-particle.

12. The method of claim 1, wherein the localizer of the first construct binds to or is coated with two or more molecules.

13. The method of claim 1, wherein the localizer binds to the first detecting material by an electrostatic, physical, chemical or biological binding.

14. The method of claim 1, wherein the label of the second construct binds to or is coated with the second detecting material directly or indirectly.

15. The method of claim 1, wherein the label of the second construct binds to the second detecting material by an electrostatic, physical, chemical or biological binding.

16. The method of claim 1, wherein one or more probe, which is selected from the group consisting of an antibody, protein, protein domain, protein motif and peptide, is used for binding the first detecting material to the localizer in the first construct, or for binding the label to the second detecting material in the second construct.

17. The method of claim 1, wherein the interaction between the first construct and the second construct is carried out in a cell.

18. The method of claim 17, wherein the cell is a prokaryotic or eukaryotic cell.

19. The method of claim 17, wherein the introduction of the first construct and the second construct into the cell is carried out by transducible peptide or fusogenic peptide, lipid or liposome, or the binding complex thereof; or by electroporation or magnetofection.

20. The method of claim 17, wherein the interaction is carried out with cells on culture plate or dish, or with microarrayed cells.

21. The method of claim 1, wherein the interaction between the first construct and the second construct is carried out in vitro.

22. The method of claim 4, wherein any material, which is translocated or not translocated according to the change of the strength of the applied magnetic field, is used as a comparative positive control or a comparative negative control, respectively.

23. The method of claim 1, wherein the detecting material of the first construct or the second construct is provided as a library.

24. A method for screening a target molecule, further comprising isolating and identifying a molecule from the first construct or the second construct, after detecting the interactions according to the method of claim 1.

25. The method of claim 24, wherein the detecting material of the first construct or the second construct is provided as a library.

26. A method for detecting molecular interactions comprising:
i) providing a first construct comprising a first detecting material and a localizer which is translocated by an externally applied driving force;
ii) providing a second construct comprising a second detecting material to which a label is attached for detection;
iii) providing a third construct comprising a material to be detected;
iv) locating the constructs, in the same field or system, near enough to interact with each other; and
v) applying an external driving force, and detecting if the label has changed in its location or motion according to the interaction.

27. The method of claim 26, wherein the detecting material of the first construct, the second construct or the third construct is provided as a library.

28. (canceled)

29. A system for detecting molecular interaction comprising:
a reactor;
a first construct comprising a first detecting material and a localizer which is translocated by an externally applied driving force;
a second construct comprising a second detecting material to which a label is attached for detection, the label changing in its location or motion when applying the external driving force according to the interaction between the first detecting material and the second detecting material; and
wherein the first and second constructs are introduced into the reactor so as to interact with each other.

30. A system for detecting molecular interaction comprising:
a reactor;
a first construct comprising a first detecting material and a localizer which is translocated by an externally applied driving force;
a second construct comprising a second detecting material to which a label is attached for detection;
a third construct comprising a material to be detected; and
wherein the first, second and third constructs are introduced into the reactor so as to interact with each other, and the label changes in its location or motion when applying the external driving force according to the interaction between the detecting materials.

31. The system of claim 29, wherein the reactor is a cell.

32. The system of claim 31, further comprising a means for detecting the change of cellular morphology or function resulted from the interaction.

33. The system of claim 29, wherein the system is manufactured in the form of a chip or a kit.

34. The method of claim 1, further comprising detecting the change of cellular morphology or function resulted from the interaction when the interaction is carried out in a cell.

35. The system of claim 30, wherein the reactor is a cell.

36. The system of claim 35, further comprising a means for detecting the change of cellular morphology or function resulted from the interaction.

37. The system of claim 30, wherein the system is manufactured in the form of a chip or a kit.

38. The method of claim 26, further comprising detecting the change of cellular morphology or function resulted from the interaction when the interaction is carried out in a cell.


SEQUENCE LISTING (3 sequences)

SEQ ID NO: 1 - DNA, 18 bp, Artificial Sequence
  Synthetic oligonucleotude having a UAS DNA sequence to which a GAL4 protein of yeast transcription factor can bind
cccagtttctagacggag

SEQ ID NO: 2 - RNA, 22 nt, Artificial Sequence
  Synthetic let-7b miRNA to bind to lin-28 mRNA
ugagguaguagguugugugguu

SEQ ID NO: 3 - DNA, 20 bp, Artificial Sequence
  Synthetic lin-28 mRNA to act as a target for let-7b miRNA
ccctatagtgagtcgtatta